(19) United States
(12) Patent Application Publication
(10) Pub. No.: US 2022/0125650 A1
(43) Pub. Date: Apr. 28, 2022

(54) SYSTEM FOR SENSING VITAL SIGNS ASSOCIATED WITH THE USE OF DISPOSABLE ABSORBENT ARTICLES

(71) Applicant: GRUPO P.I. MABE S.A. DE C.V., Puebla (MX)

(72) Inventors: Raul GONZÁLEZ MARTÍNEZ, Puebla (MX); Lucía Del Carmen SÁNCHEZ FERNÁNDEZ, Puebla (MX); Carlos CANALES ESPINOSA DE LOS MONTEROS, Puebla (MX); Alberto CORONA CARLOS, Puebla (MX)

(21) Appl. No.: 17/568,487

(22) Filed: Jan. 4, 2022

Related U.S. Application Data
(62) Division of application No. 16/546,533, filed on Aug. 21, 2019.
Application No. 16/546,533 is a continuation of application No. 15/504,517, filed on Mar. 22, 2018, now abandoned.

(30) Foreign Application Priority Data
Aug. 18, 2014 (MX) MX/A/2014/009943

(51) Int. Cl. A61F 13/84; A61B 5/00; A61B 5/02; G16H 40/67; A61B 5/0205; H04M 1/72403; A61B 90/96; A61B 5/01; A61B 5/021; A61B 5/08; A61F 13/49; A61F 13/551

(52) U.S. Cl. CPC A61F 13/84; A61F 2013/424; A61B 5/02; A61B 5/6808; G16H 40/67; A61B 5/0205; H04M 1/72403; A61B 90/96; A61B 5/002; A61B 5/01; A61B 5/021; A61B 5/0816; A61B 5/6822; A61B 5/6824; A61B 5/6833; A61B 5/6838; A61B 5/746; A61F 13/49; A61F 13/5511; A61B 2503/04; A61B 5/683; A61B 5/681; A61B 2562/08; A61B 5/0022

(57) ABSTRACT

The present invention protects a system for monitoring the vital signs of a user of a disposable absorbent article that comprises a sensor device, a bi-dimensional code and a receiver, such that the sensor device is placed in contact with the user and the system is triggered by the reading of the bi-dimensional code through the receiver, such that the bi-dimensional code is place inside the packaging of the disposable absorbent article.

### BACKGROUND OF THE INVENTION

In recent years, an infinity of proposals related to the use of computer technology associated with the use of disposable absorbent articles have been developed; such that through a sensor, any change in state of the article is detected, such as the presence of moisture and/or fecal matter, changes in pH, changes in temperature, infections, etc., the information detected by the sensor is transmitted to a receiver and said receiver sends a signal that may be transmitted and received in a mobile device, alerting the bearer of said device that such a change has occurred. In this way, the caregiver is alerted and knows when it is time to remove the article for replacing it with a new one and/or if the user of the article has undergone any change in his general health status.

The caregivers of a baby, particularly the parents, would have peace of mind and confidence to have the possibility of knowing and monitoring the vital signs, such as blood pressure, pulse, breathing rate and temperature of the baby at all times, receiving information about said vital signs on their mobile device wherever they are and continuously, as well as receiving an alarm signal in case these vital signs exceed the limits. The above also applies to the caregivers of disabled adults.

The present invention refers to a system for detecting the vital signs of a user of a disposable absorbent article, such as a diaper or disposable pants, through a device that is in contact with the user that is activated by a code or trigger mechanism associated with the article. This device detects the vital signs and sends a signal to a receiver. The receiver may be a mobile device or may receive the signal and send it to a mobile device.

As mentioned, there is a large amount of developments of sensors which are inserted inside the structure of the diaper and that are triggered by the release of urine, sending signals to a receiver which in turn transmits them to a device that alerts the caregiver and/or the user that the release has occurred. Disposable absorbent articles which include sensors or electric circuits inside its structure are described in international patent applications WO2012084924, WO2012084985 and WO2013091728 filed by SCA Hygiene Products; U.S. Pat. Nos. 7,667,608, 8,222,476, 8,264,362, U.S. Ser. No. 08/604,268, U.S. Pat. No. 8,698,641 as well as application WO2013091728 assigned to Kimberly Clark.

These types of sensors that are placed within the structure of the diaper have the significant disadvantage that may be in contact with the skin or cause discomfort for the user when triggered.

Sensors have also been developed which are not placed within the structure of the diaper. These sensors are placed outside the diaper, but remain in contact with it and may be interchangeable. This type of sensors are described in patents: U.S. Pat. Nos. 8,207,394, 8,274,393, 8,440,877, as well as in patent applications: WO2012166766, WO2013011391, WO2013076593, WO2013084085, US2013110061 and US2013110063, all filed by Kimberly Clark Corporation, as well as patent applications WO2012166766 and WO2012166765 of Procter & Gamble and application WO2013095230 filed by SCA Hygiene Products. These sensors have been developed to detect changes in the absorbent article, i.e., urine discharges, the volume thereof, pH or temperature changes due to the presence of urine or fecal matter, for which reason they must be in direct contact with the article, although they are not provided within the structure thereof.

The present invention refers to a system for detecting the vital signs of the user of a disposable absorbent article. The system foresees the use of a device that is in contact with the user, but not with the disposable absorbent article, although it is associated thereto. The device detects the vital signs of the user and monitors them at every moment, independently if the user has had any kind of urine and/or feces release onto the absorbent article. The device sends the signals wirelessly to a receiver, the receiver may be a mobile device, or it may send the data to a mobile device such that the caregiver of the baby or adult may check the status of the vital signs of the article user any time during the day, wherein the checking of the vital signs of the article user is activated by means of a code or trigger mechanism of a device linked to the disposable absorbent article.

### Objectives of the Invention

The objective of the present invention is to develop a monitoring system for vital signs in babies and adults; the system is linked to the use of a disposable absorbent article.

An additional objective of the invention is that the system uses a sensor device in contact with the user, and linked to the disposable absorbent article, but not in contact thereto.

Another objective of the invention is that the device detects the vital signs and sends the signal to a receiver.

An additional objective of the invention is that the sensor is non-invasive and may be in contact with the skin of the user for long lengths of time without causing injury to the user.

Another objective of the invention is that the detected data can be checked by the caregiver of the user of a disposable absorbent article through the receiver.

A further objective of the invention is that the system outputs an alert when the detected vital sign(s) exceed certain predetermined limits.

### BRIEF DESCRIPTION OF THE DRAWINGS

FIG. 1 illustrates the monitoring system for vital signs of the present invention.

FIG. 2a shows an example of a sensor device with a bracelet frame that could be used in the monitoring system for vital signs of the present invention.

FIG. 2b shows an example of a sensor device with a medal frame that could be used in the monitoring system for vital signs of the present invention.

FIG. 2c shows an example of a sensor device with a clip frame that could be used in the monitoring system for vital signs of the present invention.

FIG. 2d shows an example of a sensor device with a self-stick patch frame that could be used in the monitoring system for vital signs of the present invention.

FIG. 3a illustrates a QR (quick response) type of bi-dimensional code that may be used in the monitoring system for vital signs of the present invention.

FIG. 3b illustrates an Aztec type of bi-dimensional code that may be used in the monitoring system for vital signs of the present invention.

FIG. 3c illustrates a Maxicode type of bi-dimensional code that may be used in the monitoring system for vital signs of the present invention.

FIG. 3d illustrates a 49 code type of bi-dimensional code that may be used in the monitoring system for vital signs of the present invention.

FIG. 4a shows an example of a mobile telephone receiver that may be used in the monitoring system for vital signs of the present invention.

FIG. 4b shows an example of a computer receiver that may be used in the monitoring system for vital signs of the present invention.

FIG. 4c shows an example of a tablet receiver that may be used in the monitoring system for vital signs of the present invention.

FIG. 4d shows an example of a modem receiver that may be used in the monitoring system for vital signs of the present invention.

### DETAILED DESCRIPTION OF THE INVENTION

The present invention refers to a monitoring system for vital signs of a user of a disposable absorbent article such as a disposable diaper, disposable pants, feminine sanitary towel, etc.

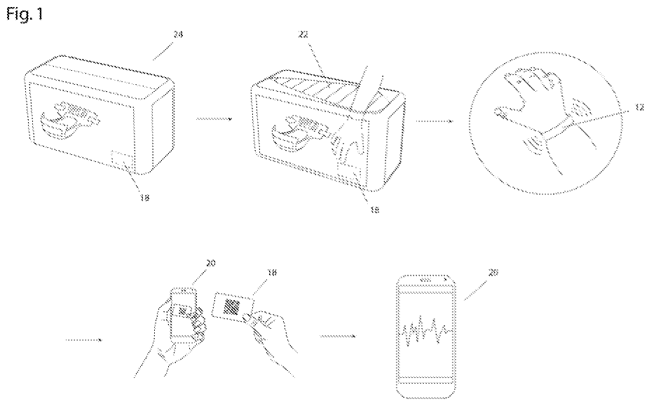

The monitoring system, shown in FIG. 1, comprises:
A sensor device (12) which is placed in contact with the user,
A code (18) or trigger mechanism of the device,
One or more disposable absorbent articles (22) placed inside a packaging (24),
a receiver (20).

The sensor device (12) is designed for measuring one or more vital signs such as pulse, temperature, breathing rate, and/or blood pressure and shall be in contact with the user. This sensor device (12) is comprised by a sensor that is placed within a frame, such as a bracelet, a necklace, a self-stick patch, a clip, etc., and is placed in contact with the user.

When placing the sensor device (12) in contact with the user, it begins to detect the vital signs. For the device to send the data to the receiver and be visualized, the device shall be triggered by a code (18) or mechanism that is placed, preferably, inside the packaging (24) of the disposable absorbent articles. Once the system is triggered, the vital signs of the user can be checked on the receiver (20), which may be directly a mobile device, or any other type of receiver that in turn, is capable of sending the signal via internet to a mobile device.

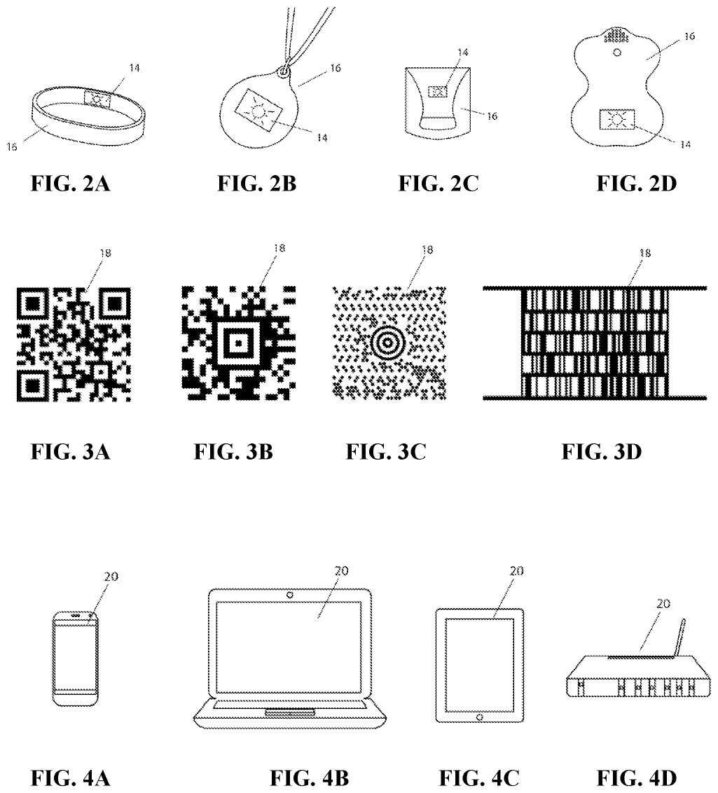

The sensor device (12) comprises a sensor (14) programmed to detect pulse, blood pressure, breathing rate, and/or temperature and a frame (16) within which said sensor is placed. FIGS. 2a to 2d illustrate different types of frames (16) that may be used along with the device (14) to form the sensor device (12). The frame may be a bracelet (FIG. 2a), a medal (FIG. 2b), a clip (FIG. 2c), a self-stick patch (FIG. 2d), or any other device that may house the sensor and that may be placed in contact with the user. In the preferred embodiment of the invention, the frame (16) is a bracelet that may be placed on the wrist or on the ankle of the user, the frame (16) is made of a material that does not injure or irritate the skin of the user of the disposable absorbent article, even he is in contact with same for long periods of time, the material can be woven or non-woven fabric, polyurethane foam, high-density polyethylene, wood, bamboo, metal, or any other type of material suitable for such ends. Further, it is desirable for the frame material to be from natural origin and/or renewable.

In case the frame (16) is a clip, it can be attached to any underwear of the user or to the disposable absorbent article (22), for instance, at the waist part of the article.

The sensor device may have a useful life of up to 3 years and is recharged by USB, replaceable batteries, rechargeable batteries, solar energy or any other power source.

The data detected by the sensor device (12) may be read through a receiver (20) which requires the system to be triggered by a bi-dimensional code (18) which is preferably placed inside the packaging (24) of the disposable absorbent articles (22). Once the system is triggered, the caregiver may check the detected data on the receiver. The entire system is illustrated in FIG. 1. The triggering of the sensor device has a service time, i.e., the receiver is capable of reading and displaying the data for a predetermined length of time, after which the system is automatically deactivated and the data may no longer be read on the receiver. The system may be activated again using a new bi-dimensional code (18) that will be obtained from a new packaging (24) of disposable absorbent articles (22). The time during which the system will be active (activation period) is associated with the read code, and this, in turn, is associated with the number of articles placed inside the bag or packaging (24), such that the system is programmed to be active during the time in which all the diapers contained in the packaging are used. For instance, in a packaging containing 12 articles, the system shall have an activation period of 4 days, considering that at least 3 diapers are used per day; thus, in a packaging of 40 articles, when reading the bi-dimensional code, the system will be activated during at least 14. The minimum activation period a code (18) will have is calculated based on the following formula:

ACTIVATION PERIOD=Integer resulting of the division of the number of articles inside the packaging/3+1

The triggering code can be any type of bi-dimensional code (18), such as bar code, QR (quick response) code, Data Matrix code, Aztec code, 49 code, Maxicode, PDF417 code, numeric, etc., as shown in FIG. 3. Preferably, the code may be read using an image reader.

The code (18) is preferably placed in the inner part of the bag or packaging (24) that contains the disposable absorbent articles. In the preferred embodiments of the invention, the code (18) is printed on the inner part of the bag or packaging (24), placed on the inner part of the by a self-stick label or may be printed onto a card, label or any other object that is placed inside the packaging (24). The code (18) may also be printed on the outer part of at least one article (22) of those that are inside the packaging (24). Finally, in another embodiment of the invention, the code (18) may also be printed on the outer part of the bag or packaging (24).

Once the caregiver opens the packaging, she/he may trigger the system through the receiver (20) using an image reader or introducing the bi-dimensional code data. The receiver (20) as illustrated in FIGS. 4a to 4b, may be a mobile telephone, a modem, a tablet, a computer or any other type of wired or wireless receiver. Upon the triggering, the caregiver will be able to check the vital signs of the user of the sensor device (12) on the receiver (20) which shall be placed within a maximum radius of 15 linear meters from the sensor device (12). The data may only be checked during the activation period, once this ends, the sensor device (12) will continue detecting the vital signs, but the caregiver will not be able to check them until he or she triggers again the system using another bi-dimensional code which can be obtained from another packaging.

Furthermore, in case the caregiver or any other person is interested in knowing the vital signs of the user of the sensor device but is located more than the previously indicated 15 linear meters radius away from the sensor device, he may access the information of the vital signs through the internet, through a mobile or electronic device, such that the caregiver or any interested adult (the parents, for example) may check the vital signs of the baby or of the adult wherever they are and at any time of the day or night.

In one embodiment of the invention, the system will output an alarm to alert the caregiver when the vital signs exceed certain predetermined limits.

The detected data can be stored the period of time that the caregiver wishes to program and may be processed in such a way that the caregiver may check the graphics of the variation of each of the detected vital sign over time.

The monitoring system for vital signs of the present invention may be associated with any article that is placed inside a packaging, preferably associated to the care of babies or incontinent adults, for example wet wipes.

While the invention has been described according to the currently preferred embodiment, it is evident that several changes or modifications thereto can be made, such changes and modifications are within the scope and spirit of the invention as defined in the appended claims.

1.-60. (canceled)

61. A process for monitoring vital signs of a user of a disposable absorbent article, comprising:
providing the user with a disposable absorbent article;
placing in contact with the user a sensor device which detects one or more vital signs data of the user;
sending the detected vital signs data from the sensor device to a receiver that is configured to read and display the detected vital signs data of the user;
accessing the vital signs data of the user through the receiver;
activating for an activation period the accessing of the vital signs data of the user with a code or mechanism that is associated with the disposable absorbent article;
wherein the vital signs data may only be accessed during the activation period.

62. The process according to claim 61, further characterized in that said sensor device comprises a sensor programed to read at least one vital sign and a frame inside which said sensor is placed.

63. The process according to claim 62, further characterized in that said sensor is programed to monitor the pulse, the breathing rate, the temperature or the blood pressure, of the user of the disposable absorbent article.

64. The process according to claim 62, further characterized in that the frame is a bracelet or a self-stick patch or a medal or a clip.

65. The process according to claim 64, further characterized in that said frame is made of a renewable or a natural origin material.

66. The process according to claim 64, further characterized in that the frame is non-woven fabric, fabric, rubber, wood, metal, bamboo, polyurethane foam, or high-density polyethylene.

67. The process according to claim 62, further characterized in that said sensor device is rechargeable through a USB, replaceable batteries, rechargeable batteries, or through sunlight.

68. The process according to claim 62, further comprising sending an alert when at least one vital sign data exceeds a predetermined limit.

69. The process according to claim 61, further characterized in that the code is a bi-dimensional code and is a QR code, a Data Matrix code, an Aztec code, a 49 code, a Maxicode code, a PDF417 code or a numeric code.

70. The process according to claim 69, further characterized in that the disposable absorbent article is provided inside a packaging and the bi-dimensional code is printed inside the disposable absorbent article packaging or is printed on any object that is placed inside the disposable absorbent article packaging.

71. The process according to claim 70, further characterized in that the bi-dimensional code is printed on an outer part of at least one disposable absorbent article that is placed inside the packaging.

72. The process according to claim 69, further characterized in that that the disposable absorbent article is provided inside a packaging and the bi-dimensional code is printed on an outer part of the packaging of the disposable absorbent article.

73. The process according to claim 61, further characterized in that the disposable absorbent article is provided inside a packaging comprising a plurality of disposable absorbent articles and the activation period is calculated according to the number of disposable absorbent articles inside the packaging using the following formula:
activation period=Integer resulting of the division of the number of articles inside the packaging/3+1.

74. The process according to claim 61, further characterized in that the receiver is a cell phone with an image reader, a tablet, a modem or a computer.

75. The process according to claim 61, further characterized in that the receiver is capable of reading and displaying the vital signs data of the user of the article within a maximum radius of 15 meters of the sensor device.

76. The process according to claim 61, further comprising uploading the vital signs data of the user through the receiver to the internet, and wherein the accessing of the vital signs data is through the internet.

77. The process according to claim 61, wherein the activating comprises introducing the code or mechanism to the receiver.

78. The process according to claim 61, wherein the sensor device continues to detect one or more vital signs data of the user after the activation period.

79. A process for monitoring vital signs of a user of a disposable absorbent article comprising:
providing the user with a packaging comprising a plurality of disposable articles and a bi-dimensional code;
placing in contact with the user a sensor device that detects one or more vital signs data of the user; wherein the sensor device comprises a sensor programmed to read at least one vital sign and a frame within which said sensor is located;
sending the detected vital signs data of the user to a receiver that is configured to read and display the detected vital signs data of the user;
accessing the vital signs data of the user through the receiver;
activating for an activation period the accessing of the vital signs data of the user with the bi-dimensional code;
wherein the vital signs data may only be accessed during the activation period;
wherein the activation period is determined in the bi-dimensional code.
